(19) United States
(12) Patent Application Publication
(10) Pub. No.: US 2013/0012902 A1
(43) Pub. Date: Jan. 10, 2013

(54) WOUND DRESSING COMPRISING A SUPERABSORBING SUBSTANCE

(75) Inventor: Rolf Rovaniemi, Rimforsa (SE)

(73) Assignee: VIR I KINDA AB, RIMFORSA (SE)

(21) Appl. No.: 13/581,136

(22) PCT Filed: Feb. 28, 2011
(86) PCT No.: PCT/EP2011/052948

§ 371 Date: Sep. 25, 2012

(30) Foreign Application Priority Data
Feb. 26, 2010 (EP) PCT/EP2010/052508

(51) Int. Cl. A61F 13/537; A61F 13/512; A61F 13/534

(52) U.S. Cl. 604/369; 604/378; 604/374; 604/367; 604/383

(57) ABSTRACT

A wound dressing can include a backing layer, a facing layer which is permeable for wound exudate and blood and which, when in use, is in contact with the wound, and an absorbent core. The backing layer and the facing layer can be joined together in order to form a pouch. The absorbent core can be located within the pouch and can include at least a first layer and a second layer. The first layer can be located closer to the facing layer than the second layer. The first layer can be configured to provide a flow of liquid predominantly in a direction perpendicular to the extension of the facing layer. The second layer can be configured to provide a flow of liquid predominantly in a direction parallel to the extension of the facing layer.

### CROSS-REFERENCE TO RELATED APPLICATIONS

This application is the national stage of PCT/EP2011/052948 filed Feb. 28, 2011, designating, inter alia, the United States and claiming priority to Patent Application No. PCT/EP2010/052508 filed Feb. 26, 2010, each of which is hereby incorporated by reference.

### BACKGROUND

Clinical use of such wound dressing has shown that the skin surrounding wounds treated by wound dressings of the above design tends to show maceration, i.e. a softening and whitening of skin which is constantly kept wet. The wound dressings according to the prior art do not achieve an optimum moisture balancing in order to support the healing process in the wound bed.

### BRIEF SUMMARY

One or more embodiments disclosed within this specification relate to a wound dressing. The wound dressing can include a backing layer, a facing layer which is permeable for wound exudate and blood and which, when in use, is in contact with the wound, and an absorbent core. The backing layer and the facing layer can be joined together in order to form a pouch. The absorbent core can be located within the pouch and can include at least a first layer and a second layer. The first layer can be located closer to the facing layer than the second layer. The first layer can be configured to provide a flow of liquid predominantly in a direction perpendicular to the extension of the facing layer. The second layer can be configured to provide a flow of liquid predominantly in a direction parallel to the extension of the facing layer.

### BRIEF DESCRIPTION OF THE SEVERAL VIEWS OF THE DRAWINGS

FIG. 1 shows a schematic drawing of a wound dressing in accordance with one embodiment disclosed within this specification.

FIG. 2 shows a tube design of a wound dressing in accordance with one embodiment disclosed within this specification.

FIG. 3 shows an alternative tube design of a wound dressing in accordance with one embodiment disclosed within this specification.

FIG. 4 shows a sandwich design of a wound dressing in accordance with one embodiment disclosed within this specification.

FIG. 5 shows a schematic drawing of a wound dressing according to a first embodiment of the present invention for moderate or slightly secreting wounds.

FIG. 6 shows a schematic drawing of a wound dressing according to a further embodiment of the present invention for heavily secreting wounds.

FIG. 7 shows a schematic drawing of a wound dressing according to a further embodiment of the present invention for secreting and blooding wounds.

FIG. 8 shows a schematic drawing of a wound dressing according to a further embodiment of the present invention for heavily secreting wounds.

FIG. 9 shows a schematic drawing of a wound dressing according to a further embodiment of the present invention for blooding wounds.

FIG. 10 shows a schematic drawing of a wound dressing according to a further embodiment of the invention having a perforated second layer.

FIG. 11 shows a schematic drawing of a further embodiment of a wound dressing in accordance with one embodiment disclosed within this specification.

### DETAILED DESCRIPTION

The arrangements described herein relate to a wound dressing comprising a backing layer, a facing layer which is permeable for liquid substances, for example wound exudate and blood, and which when in use is in contact with the wound, and an absorbent core, wherein the backing layer and the facing layer are joined together in order to form a pouch, and wherein the absorbent core is located within the pouch, wherein the absorbent core comprises at least a first and a second layer, wherein the first layer is located closer to the facing layer than the second layer, wherein the first layer is arranged such that when in use it provides a flow of liquid predominantly in a direction perpendicular to the extension of the facing layer, and wherein the second layer is arranged such that when in use it provides a flow of liquid predominantly in a direction parallel to the extension of the facing layer. The wound dressing avoids maceration in a wound and achieves a balanced moisture in the wound bed.

It is assumed that at least the facing layer is a laminar structure whose thickness is small compared to its length and width. As such the extension of the facing layer denotes its laminar extension. A direction parallel to the extension of the facing layer is essentially parallel to a plane defined by the width and length of the facing layer. A direction perpendicular to the extension of the facing layer is essentially parallel to the thickness of the facing layer.

The basic concept of the design of the absorbent core according to an embodiment of the present invention having two distinct layers is to allow for a quick and effective transport of liquid, in particular exudate and blood, from the wound while storage of liquids is established in a predetermined distance from the actual wound surface.

In order to achieve such functionality the absorbent core is formed by at least two layers. The first layer is arranged such that it predominantly provides a transport of liquid in a direction perpendicular to the facing layer, i.e. in a direction away from the wound.

The first layer also serves as a distance element. It provides a distance between the actual wound and the second layer which serves as a storage for liquid sucked out of the wound. Thus the stored liquid does not contribute to maceration of the skin surrounding the wound.

In order to achieve a flow of liquid predominantly in a direction perpendicular to the facing layer in a first embodiment the first layer consists of synthetic material only.

A particular example for a synthetic material for forming the first layer is dry polyester textile made of hydrophilic PET and bicomponent fibers bonded to form a web as it is for example available from Libeltex of Meulebeke, Belgium under the tradename DRY WEB T1.

In particular in such an embodiment, in which the first layer consists of synthetic material only it is advantageous once the first layer is free of any superabsorbent substance. In an alternative embodiment the first layer may contain a small content of superabsorbent material having weight in a range from 10 g/m² to 40 g/m².

In an embodiment in which the first layer is made of synthetic material only and being free of superabsorbent materials or only having a low content of superabsorbent material the first layer provides a moisture balancing of the wound and a distribution of exudates from the wound.

If however it is preferred to have some absorption capability of the first layer as well it will be advantageous once the first layer is either made of a mixture of synthetic fibers and cellulose based material or of cellulose based material only.

Whenever the term cellulose based material is used within this application it particularly refers to for example cellulose, tissue paper, cotton, viscose or a mixture of virgin defibrated cellulose and tissue paper. However, other materials based on cellulose are well included by the term cellulose based material.

In an embodiment of the invention it is preferable once the first layer in its un-wetted state has a weight in a range from 12 g/m² to 150 g/m² and preferably in a range from 40 g/m² to 100 g/m².

Furthermore in an embodiment the first layer in its un-wetted state has a density in a range from 0.03 g/m² to 0.4 g/m².

In order to achieve a flow of liquid predominantly in a direction perpendicular to the facing layer in an alternative embodiment the first layer may comprise a superabsorbent substance.

When entering the first layer water or more general the liquid parts of the secretions absorbed from the wound is trapped by the superabsorbent substance contained in the first layer. This trapping in the superabsorbent substance will lead to an effective blocking of liquid transport in a direction parallel to the facing layer, i.e. in the plane of the first layer of the absorbent core.

In order to avoid an entire blocking of liquid transport not only in a direction parallel to the facing layer, but also in a direction perpendicular to the facing layer, i.e. in a direction in which effective transport of liquid sucked from the wound is required in order to maintain the functionality of the wound dressing, the first layer in an embodiment contains cellulose or viscose or a mixture of cellulose and viscose.

To establish a flow of liquid predominantly in a direction perpendicular to the facing layer in an embodiment of the invention the superabsorbent substance in the fibers of the first layer can be particularly distributed to establish at least a section of the first layer having a lower density of the superabsorbent substance than the adjacent sections of the first layer such that a channel perpendicular to the extension of the facing layer is formed. In the channel the density of the superabsorbent substance may be zero.

In a further embodiment the first layer comprises a tissue paper or defibrated virgin cellulose fibers or a mixtures of both and a superabsorbent substance, wherein the weight of the tissue paper or the defibrated virgin cellulose fibers or a mixture of both in its un-wetted state is in a range from 20 g/m² to 300 g/m², preferable in a range from 40 g/m² to 100 g/m², and wherein the weight of the superabsorbent substance in its un-wetted state is in a range from 10 g/m² to 100 g/m², preferable in a range from 20 g/m² to 60 g/m². The contents of cellulose or viscose in the first layer enables a liquid transport in direction perpendicular to the facing layer even if the superabsorbent polymers contained in the first layer are already saturated. I.e. the cellulose or viscose avoid a blocking of liquid transport in a direction perpendicular to the facing layer.

In an embodiment the first layer in addition contains a fraction of synthetic fibers, preferably 0.1% by weight to 10% by weight, and most preferred 1% by weight to 5% by weight, of synthetic fibers. Adding synthetic fibers to a tissue paper or defibrated virgin cellulose fibers or a mixtures of both does stabilize the first layer.

Whenever in the context of the present application a weight is given in grams per square meter (g/m²) it means that a square meter of the respective material weights to the amount of grams given. This weight in the paper industry is commonly denoted as paper weight, wherein the units grams per square meter is identical to gsm. It is commonly named an area weight. When within this application a weight of superabsorbentpolymers is given in grams per square meter (g/m²) it denotes the area weight of the superabsorbent polymers when taken on their own without fibers. Whenever a weight is given in the context of this application it is given for the material in its un-wetted state.

Superabsorbent substances in the sense of the present application are materials that have the ability to absorb and retain large volumes of water and aqueous solutions. Superabsorbent substances falling into this category are for example modified starch, polymer like polyvinyl alcohol (PVA), polyethylene oxide (PEO) which are all hydrophilic and have a high affinity for water. When lightly chemically or physically cross-linked, these polymers are water-swellable but not water-soluble. The afore-mentioned superabsorbents have been known for a long time.

In a more preferred embodiment the superabsorbent substance in the sense of the present application is a superabsorbent polymer made from partially neutralized, lightly cross-linked polyacrylic acid, which has been proven to give the best performance vs. cost ratio. Those superabsorbent polymers in an embodiment are manufactured at low solids levels for quality economic reasons and are dried and milled into granular white solids. In water they swell to a rubbery gel. Superabsorbent polymers may absorb up to 500 times their weight of water.

In an alternative embodiment the different functionalities of the first and second layer with respect to their predominant liquid transport properties may be achieved by choosing the material for the first layer such that it has a lower density than the material of the second layer. In such an embodiment the first layer may alternatively contain or not contain a superabsorbent substance.

Alternately the first and second layer can be integrally formed having a gradient of density with the lowest density close to the facing layer and a higher density at the bottom furthest away from the facing layer.

In an embodiment in which the first layer of the absorbent core does not comprise a superabsorbent polymer the weight of the cellulose based material used to form the first layer in its un-wetted state is in a range from 20 g/m² to 400 g/m², preferable in a range from 80 g/m² to 200 g/m².

Although the core may comprise of at least two layers differing from each other with respect to their liquid transport properties, this does not exclude the presence of further layers. In particular the core may in an embodiment have four or six layers in order to create an improved fluid distribution. Moreover the first and second layers in an embodiment may be formed of a plurality of sub-layers.

The second layer does provide a transport of fluid over the whole core surface in a direction predominantly parallel to the extension of the facing layer and serves as a storage for the fluid.

In order to achieve that the second layer of the absorbent core provides a flow of liquid predominantly in a direction parallel to the facing layer the second layer in a first embodiment of the present invention comprises a first sub-layer and a second sub-layer, wherein the first sub-layer of the second layer is closer to the first layer than the second sub-layer of the second layer, wherein the first sub-layer comprises a cellulose based material, and wherein the second sub-layer comprises a cellulose based material, preferably a fibrous material based on cellulose, and a superabsorbent material.

In such a design it is particularly useful once the first sub-layer in its un-wetted state has a weight in a range from 40 g/m² to 400 g/m², wherein the superabsorbent material of the second sublayer (6b) in its un-wetted state has a weight in a range from 40 g/m² to 500 g/m², preferably 50 g/m² to 200 g/m² and wherein second sub-layer including the superabsorbent material in its un-wetted state has a weight in a range from 100 g/m² to 800 g/m², preferably in a range from 200 g/m² to 500 g/m².

The term sub-layer in the sense of the present application denotes a layer which together with one or more other sub-layers provides a functional or manufacturing unit. In particular the at least two sub-layers of the second layer do together provide the functionality of a flow of liquid in a direction predominantly parallel to the extension of the facing layer. Furthermore the first and the second sub-layer of the second layer may be manufactured as a unit. I.e. the first and second sub-layers may be pre-manufactured and introduced into the actual production line together. However, from a physical point of view the sub-layers of the second layer are independent layers and the first and second layers in this embodiment of the invention could alternatively be described as three different layers. Thus any device will fall into the scope of protection as claimed once it provides three different layers with the claimed design and functionality.

In addition the first and second sub-layers of the second layer may in an embodiment comprise at least one further sub-layer. Once the second layer comprises a further sub-layer it is preferred if in an embodiment the third sub-layer has identical properties and design as the second sub-layer. By providing further sub-layers an anti-slumping effect is provided as well as an enhancement of the flow properties of the second layer in a direction parallel to the extension of the facing layer.

In an embodiment the second to n-th sub-layers are chosen such that together they do provide the properties of a second sub-layer of an embodiment just having two sub-layers in the second layer. Thus in an embodiment the superabsorbent material in the second to n-th sub-layers in its un-wetted state has a weight in a range from 40 g/m² to 500 g/m², preferably 50 g/m² to 200 g/m² and wherein second sub-layer to n-th sub-layer including the superabsorbent material in its un-wetted state have a weight in a range from 100 g/m² to 800 g/m², preferably in a range from 200 g/m² to 500 g/m².

In a further embodiment the first layer is perforated by a plurality of holes. Those holes preferably do have a diameter in a range from 1.5 mm to 5 mm, preferably in a range from 2 mm to 4 mm. In an embodiment the surface area of each hole is in a range from 1.8 mm² to 20 mm², preferably in a range from 3.14 mm² to 13 mm² This enhances the fluid transport in the first layer in a direction perpendicular to the extension of the layer.

In addition or alternatively the moisture balance layer and/or the facing layer is perforated by a plurality of holes, wherein the holes preferably do have a diameter in a range from 1.5 mm to 5 mm, preferably in a range from 2 mm to 4 mm.

Cellulose or viscose or a combination of both can be used either to manufacture paper tissue or to manufacture a non-woven fabric.

A non-woven fabric in the sense of the present application is a material made of at least one layer of long fibers which have been formed to a web and in a next step consolidated. In particular the consolidation of a non-woven fabric may be achieved by friction and/or cohesion and/or adhesion, for example by needling, felting, spun lacing or melting.

If compared to tissue paper a material will be considered a non-woven fabric in the sense of the present application if more than 50% of the mass of its fibers components consists of fibers having a ratio of a lengths to their diameter of more than 300. Alternatively, the material will be considered a non-woven fabric in the sense of the present application if this condition is not fulfilled but if more than 30% of the mass of its fibrous components consist of fibers having a ratio of their lengths to their diameter of more than 300 and its density is lower than 0.4 g/cm³. This definition corresponds to EN 29 092.

In a further embodiment the wound dressing according to an embodiment of the present invention comprises a moisture balance layer located between the absorbent core and the facing layer, wherein the moisture balance layer comprises a non-woven fabric consisting of synthetic or natural fibers such as cellulose fibers. In an embodiment the fibers of the non-woven fabric in the moisture balance layer are orientated such that they predominantly extent in a direction perpendicular to the extension of the moisture balance layer as well as to the extension of the other layers.

The moisture balance layer is thought to improve the moisture balancing of the wound and can be alternatively used as a blood absorption layer when used in dressings applied to heavily secreting or blooding wounds.

In an embodiment the moisture balance layer in its un-wetted state comprise a density in a range from 0.04 g/cm³ to 0.4 g/cm³.

In a further embodiment the fibers of the moisture layer in its un-wetted state comprise a weight in a range from 5 g/m² to 200 g/m² and preferably in a range from 12 g/m² to 150 g/m².

It is particularly useful if the fibers of the moisture balance layer in its un-wetted state comprise a weight in a range from 18 g/m² to 80 g/m² and the moisture balance layer further comprises a superabsorbent substance which in its un-wetted state comprises the weight in a range from 10 g/m² to 30 g/m².

The moisture balance layer or the blood absorption layer is an additional layer provided in addition to the absorbent core with its first and second layers.

In an embodiment the facing layer is made of a material consisting of one selected of a group comprising a non-woven fabric, a perforated film and a foam based on polyurethane or silicon or a combination thereof.

In a further embodiment the facing layer comprises a non-woven fabric consisting of synthetic or cellulose fibers, wherein the fibers of the non-woven fabric are orientated such that they predominantly extend in a direction perpendicular to the extension of the facing layer. Such orientation of the fibers in the non-woven fabric is achieved by orienting the fibers during the fabrication process, in particular during spun lacing and needling

In an embodiment of the invention the facing layer comprise a density in a range from 0.1 g/cm³ to 0.6 g/cm³.

In a further embodiment the facing layer in its un-wetted state comprise a weight in a range from 12 g/m² to 100 g/m², preferably in a range from 18 g/m² to 70 g/m².

It is further useful if in an embodiment the facing layer comprises a hydrophobic or hydrophilic and/or bacteriocidal or bacteriostatic agents.

In an embodiment of the present invention the backing layer is made of a breathable non-woven fabric or perforated film.

The pouch made of the facing layer and the backing layer can be fabricated in different ways. If the facing layer and the backing layer consist of an identical material a single sheet of material can be used, folded and sealed together at its edges in order to form the pouch.

Alternatively two single sheets, one forming the facing layer, one forming the backing layer can be connected to each other at their edges in order to form the pouch accommodating the absorbent core.

Alternatively the facing layer and the backing layer may be connected to each other in order to form a tubular structure. Therefore to opposite edges of the facing layer are folded, such that in its cross section a C-shaped structure is formed and the backing layer is connected to the two folded ends of the facing layer.

In an embodiment of the invention the absorbent core comprises active wound healing substances, like for example arnica montana, polyhexanide (PHMB) polyhexamethylene or chitosan which are released when the wound dressing is applied to a patient's wound.

Further advantages, features and applications of the present invention will be apparent from the following description of embodiments and the related figures. In the following description of the figures elements having equivalent functionality are denoted by same reference numbers.

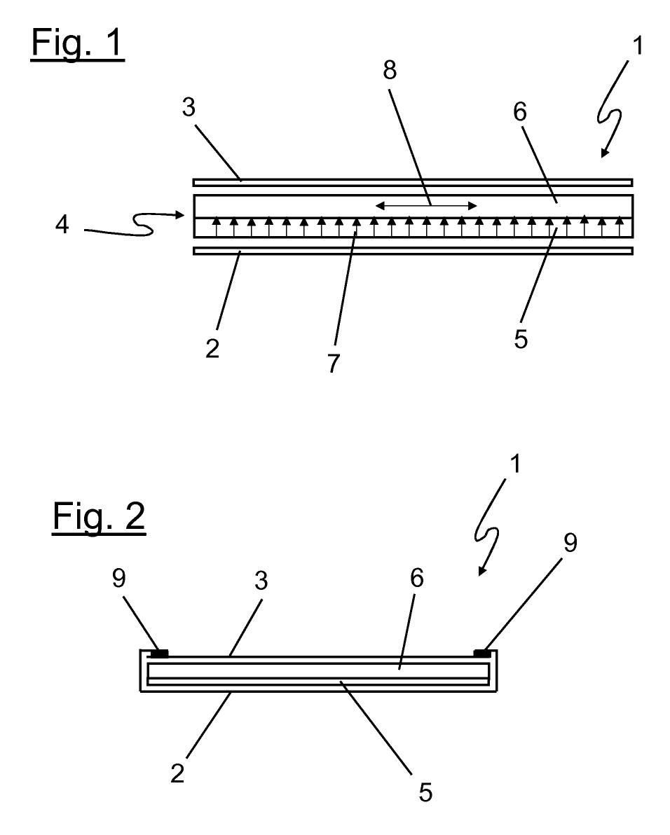

FIG. 1 shows a schematic drawing of a wound dressing 1 in accordance with one embodiment disclosed within this specification. FIG. 1 is thought to schematically describe an aspect of the present invention. The wound dressing is formed by a facing layer 2 which when in use is brought into contact with the patient's wound and by a backing layer 3.

The facing layer 2 and the backing layer 3 are joined together (not shown in FIG. 1) in order to form a pouch in which an absorbent core 4 is contained. In order to allow for an effective transport of exudates from the patient's wound the facing layer 2 is made of a non-woven fabric consisting of synthetic fibers with an open structure.

The backing layer 3 in the embodiment depicted in FIG. 1 is made of a perforated film allowing to keep the moisture in the dressing 1 while being breathable through the perforations of the film. In an alternative design the backing layer me be formed of a breathable film.

The absorbent core 4 consists of a first layer 5 and a second layer 6. The first layer 5 is arranged such that when in use it provides a flow of liquid predominantly in a direction perpendicular to the extension of the layers 2, 3, 5, 6, in particular to the facing layer 2. This is indicated by the arrows 7 in the first layer 5. In contrast the second layer 6 is arranged such that when in use it provides a flow of liquid predominantly in a direction parallel to the layers 2, 3, 5, 6, in particular parallel to the facing layer 2. This is indicated by arrow 8 in layer 6.

In order to achieve the preferred directions for the transport of liquid in the two core layers 5, 6 the first layer 5 which is located closer to the facing layer 2 than the second layer 6 contains a superabsorbent polymer, for example a superabsorbent polymer available under the trademark Favor from Evonik Stockhausen GmbH.

When a certain amount of liquid has been absorbed by the first layer 5 the gel formed by the superabsorbent polymer trapping the liquid effectively blocks a liquid transport in a direction parallel to the extension of the facing layer 2 such that the transport process in the first layer 5 predominantly occurs in a direction perpendicular to the extension of the facing layer 2, i.e. towards the second layer 6.

In contrast the second layer 6 or in particular the first sub-layer in the second layer does not contain any superabsorbent material at all such that when in use it provides a liquid flow in a direction essentially parallel to the extension of the facing layer 2. In an alternative design (not shown) the second layer, in particular the first sub-layer in the second layer, may comprise cellulose in a fluff form.

In the example depicted in FIG. 1 the structure of the first layer 5 is made of a tissue paper of cellulose and viscose fibers in which the superabsorbent polymer is embedded. The second layer also consists of a tissue paper. In order to stabilize the first layer 5 it contains 3% by weight of synthetic fibers.

FIGS. 2 to 4 do show different types of designs for the pouch formed by the backing layer 3 and the facing layer 2.

Each of the three wound dressings depicted in FIGS. 2 to 4 do contain an absorbent core 4 having a first layer 5 and a second layer 6. The first and second layers 5, 6 are designed as described in detail with respect to the embodiment depicted in FIG. 1.

FIG. 2 shows a tube design in which the facing layer 2 has been folded twice at opposing edges such that the facing layer 2 partly extends over the backside of the absorbent core 4. The backing layer 3 has been sealed with seals 9 to the sections of the facing layer extending over the backside of the core 4, wherein the facing layer 2 overlaps the backing layer 3. In the embodiment of FIG. 2 the backing layer is located below the folded edges of the facing layer 2.

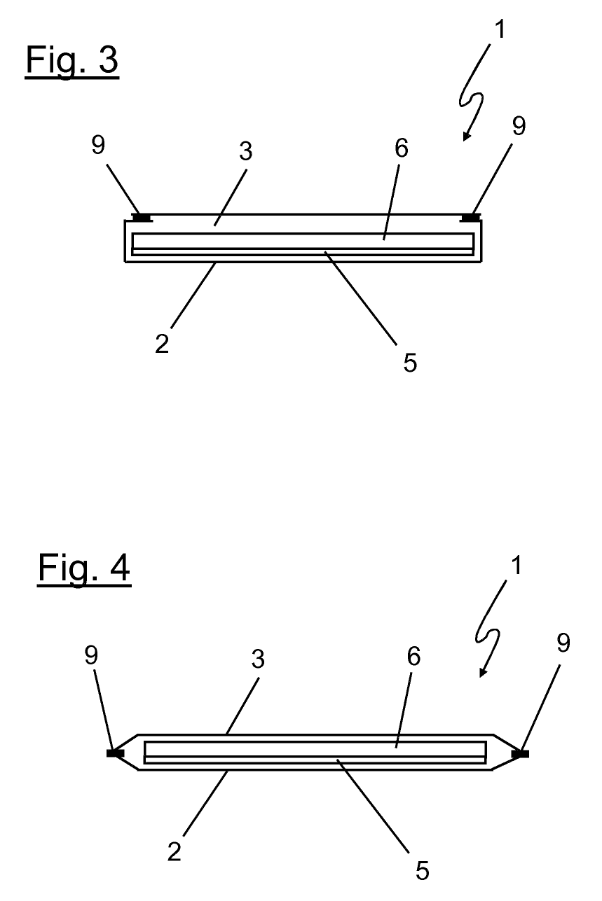

FIG. 3 shows an alternative embodiment of the tube design as depicted in FIG. 2. The construction of the dressing has been turned around with respect to the order of the facing layer 2 and the backing layer 3 at the backside of the core in the area of the seals 9. In this particular embodiment the backing layer is located on top of the folded edges of the facing layer 2.

FIG. 4 shows a sandwich design in which the two sheets forming the facing layer 2 and the backing layer 3 have been laid on top of each other and the seals 9 connecting the edges of the facing layer 2 and the backing layer 3 enclose the core 4.

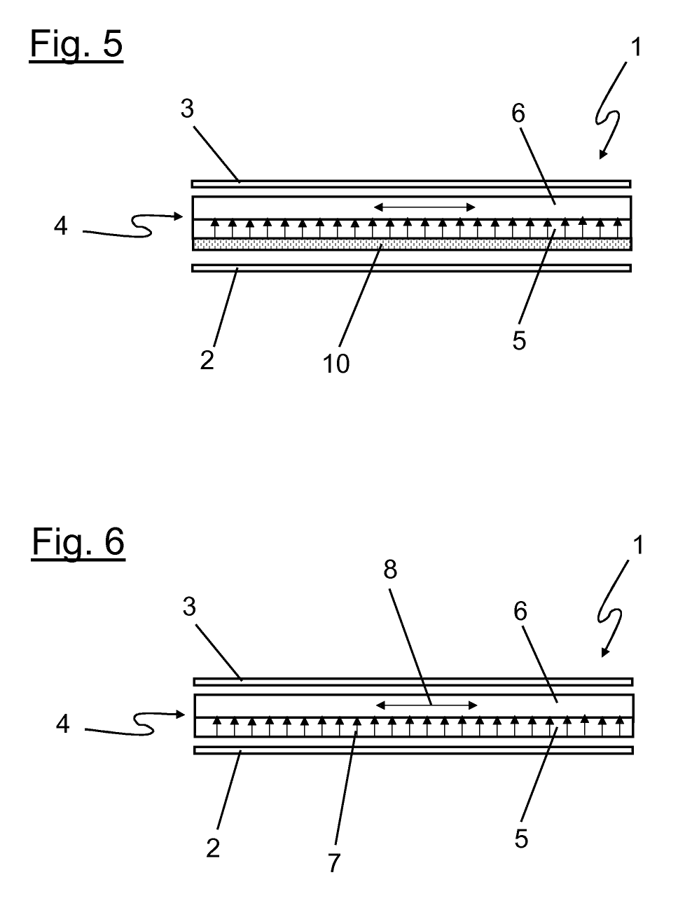

While not depicted in the figures in any of the embodiments according to the following FIGS. 5 to 10 the backing layer 3 and the facing layer 2 are joined together in order to form a pouch. The design of the pouch may for example be chosen from any of the designs depicted in FIGS. 2 to 4.

FIG. 5 shows a wound dressing according to an aspect of the present invention for modestly or slightly secreting wounds. The backing layer 3 consists of a non-woven fabric comprising a hydrophobic treatment.

The facing layer 2 in the embodiment according to FIG. 5 consists of a foam based on polyurethane providing good vertical fluid distribution properties. The facing layer 2 further comprises an anti-bacterial and hydrophobic treatment in order to enhance its liquid transport properties and to avoid any bacterial infections on the wound surface.

Alternatively to what has been shown in FIG. 5 the facing layer 2 may be formed as a fluid distribution layer comprising a mixture of synthetic, natural and cellulose fibers, wherein the fibers have a predominant orientation, such that the majority of the fibers can be oriented in a direction perpendicular to the facing layer 2. In general the density of the facing layer 2 in this case could be in a range from 0.1 g/cm³ to 0.6 g/cm³ having a weight in a range from 12 g/m² to 100 g/m².

The absorbent core 4 of the embodiment depicted in FIG. 5 comprises a first layer 5 and a second layer 6. The second layer 6 consists of defibrated virgin cellulose fibers having a weight per area of 200 g/m².

The second layer 6 is designed in order to provide a preliminarily horizontal fluid distribution in the layer. In contrast the first layer 5 in the core 4 consists of defibrated virgin cellulose fibers, commonly denoted as fluff, in their un-wetted state having a weight per area of 200 g/m². The first surface layer in order to provide a preliminarily vertical, i.e. perpendicular to the extension of the facing layer 2, transport of liquid contains a superabsorbent polymer based on acrylic acid. The absorbent polymer in its un-wetted state has weight per area of the surface of the overall layer 5 of 150 g/m².

In addition to the absorbent core 4 a moisture balance layer 10 is located between the core 4 and the facing layer 2. The moisture balance layer 10 consist of a mixture of synthetic and natural fibers, wherein in the present case the natural fibers are made of cellulose. In order to allow for a moisture balancing the amount of synthetic fibers is about 50% of the overall amount of fibers in the moisture balance layer. The fiber in the moisture balance layer 10 have been oriented vertically, i.e. essentially perpendicular to the extension of the facing layer 2, during the needle punching process of the non-woven fabric forming the moisture balance layer 10. In the embodiment depicted the fibers of the non-woven fabric forming the moisture balance layer 10 do have a density of 0.04 g/cm³ and the non-woven fabric in its un-wetted state has a weight per area of 80 g/m².

In an alternative embodiment (not depicted in FIG. 5) the non-woven fabric of the moisture balance layer 10 may carry a superabsorbent polymer based on acrylic acid having a weight in a range from 10 g/m² to 30 g/m². Alternatively a superabsorbent polymer may be arranged between two of the above mentioned non-woven fabric layers which then together do form the moisture balance layer. In this case the weight of the superabsorbent polymer in its un-wetted state is in a range from 30 g/m² to 80 g/m².

In the embodiment shown in FIG. 5 the facing layer 10 additionally comprises an active substance, in this case Arnica Montana, accelerating the healing process.

FIG. 6 shows a design of a wound dressing according to an aspect of the present invention for application to heavily secreting wounds. As in FIG. 1 the wound dressing according to FIG. 6 only shows a pouch formed by a backing layer 3 and a facing layer 2, wherein in the pouch a core 4 comprising a first layer 5 and a second layer 6 is located.

As described before with reference to FIG. 5 the backing layer 3 consists of a non-woven fabric having a hydrophobic treatment.

The facing layer of 2 of the embodiment shown in FIG. 6 consists of a hydrophilic layer of a foam made of polyurethane which in its un-wetted state comprises a weight per square meter of the area of the facing layer 2 in a range from 100 g/m² to 600 g/m².

While this weight per area of the facing layer has been chosen for the particular embodiment depicted in FIG. 6 in general a spun bond non-woven used as a facing layer in a dressing as depicted in FIG. 6 in its un-wetted state may have a weight per area in a range from 12 g/m² to 40 g/m². Alternatively the facing layer 2 may be formed of an spundbond-meltblown-spunbond (SMS) non-woven having a weight per area in its un-wetted state in a range from 12 g/m² to 30 g/m². Even a spundbond-meltblown-spunbond-meltblown-spunbond (SMSMS) non-woven having a weight per area in its un-wetted state in a range from 6 g/m² to 20 g/m² could be used for forming the facing layer 2.

Alternatively the facing layer 2 of the embodiment depicted in FIG. 6 could consist of a synthetic fiber which in a needle punching process has been treated to form a non-woven fabric having a density of the layer in a range from 0.2 g/cm³ to 0.6 g/cm³ and a weight per area in its un-wetted state in a range from 20 g/m² to 70 g/m².

In an alternative design the facing layer may comprise an alginate.

The absorbent core 4 again consists of a first layer 5 and a second layer 6. The first layer which provides a predominantly vertical fluid distribution, i.e. in a direction perpendicular to the extension of the facing layer 2, consists of tissue paper having a weight per area of 100 g/m², wherein in the tissue paper a superabsorbent polymer based on acrylic acid is integrated having a weight per area of 200 g/m2.

Again the second layer 6 of the core 4 provides a predominantly horizontal, i.e. essentially parallel to the extension of the facing layer 2, fluid transport. The second layer 6 of the core 4 of the embodiment depicted in FIG. 6 consists of tissue paper having a weight of 200 g/m².

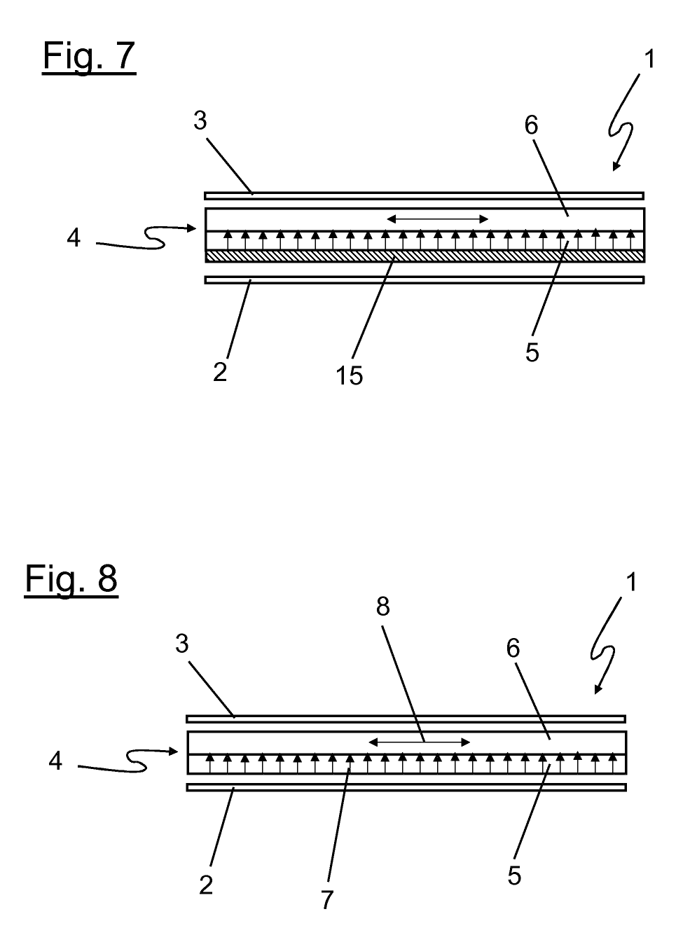

The embodiment depicted in FIG. 7 also shows a wound dressing for heavily secreting wounds which provides an additional blood absorption layer 15. The construction of the other layers of the dressing, i.e. the backing layer 3, the facing layer 2 and the absorbent core 4 with its first layer 5 and its second layer 6 are identical to the embodiment depicted in FIG. 6.

The additional blood absorption layer 15 consists of a mixture of synthetic fibers and cellulose fibers which have been oriented vertically, i.e. essentially perpendicular to the extension of the facing layer 2, during the spun laced or needle punched process used to manufacture the nonwoven fabric forming the blood absorption layer 15. The blood absorption layer 15 in the embodiment depicted in FIG. 7 does have a density of 0.04 g/cm³ and in its un-wetted state the non-woven fabric of the blood absorption layer 15 has a weight per area in a range from 30 g/m² to 150 g/m². In order to provide an effective blood absorption the blood absorption layer 15 has a contents of more than 80% of natural fibers, in particular cellulose fibers.

FIG. 8 shows an alternative embodiment for a wound dressing for heavily secreting wounds. The design is most comparable to what has been with respect to the embodiment of FIG. 6, i.e. the dressing comprises facing layer 2, a backing layer 3 and a core 4 having a first layer 5 and a second layer 6. Additional moisture balancing or blood absorption layers need not be present.

While the backing layer 3 and the facing layer 2 have been chosen as in the design according to FIG. 6 the core layers 5, 6 have been designed not only to provide a predominantly vertical (first layer 5) or predominantly horizontal (second layer 6) fluid transport, absorption and retention but also to provide an anti-slumping effect and to avoid gel blocking in order to create a better fluid distribution.

In order to achieve those objects the first layer 5 and the second layer 6 each consist of virgin cellulose fibers having a weight per area of 80 g/m². Each of the layers 5, 6 contains super absorbent polymers based on acrylic acid with a weight per area of 170 g/m² in the virgin cellulose fibers.

In order to strengthen the anti-slumping effect and fluid transport the second layer 6 can be divided into a plurality of sub-layers (not depicted tin FIG. 8). The number of sublayers forming the second layer 6 may range from two to four sub-layers. Each of the sub-layers may contain a weight ratio between cellulose and superabsorbent polymers in a range from 1.5 to 1. In an embodiment the total amount of cellulose when added over all sub-layers is 600 g/m² and the total amount of superabsorbent polymers when added over all sub-layers is then 500 g/m² divided over two to four sub-layers.

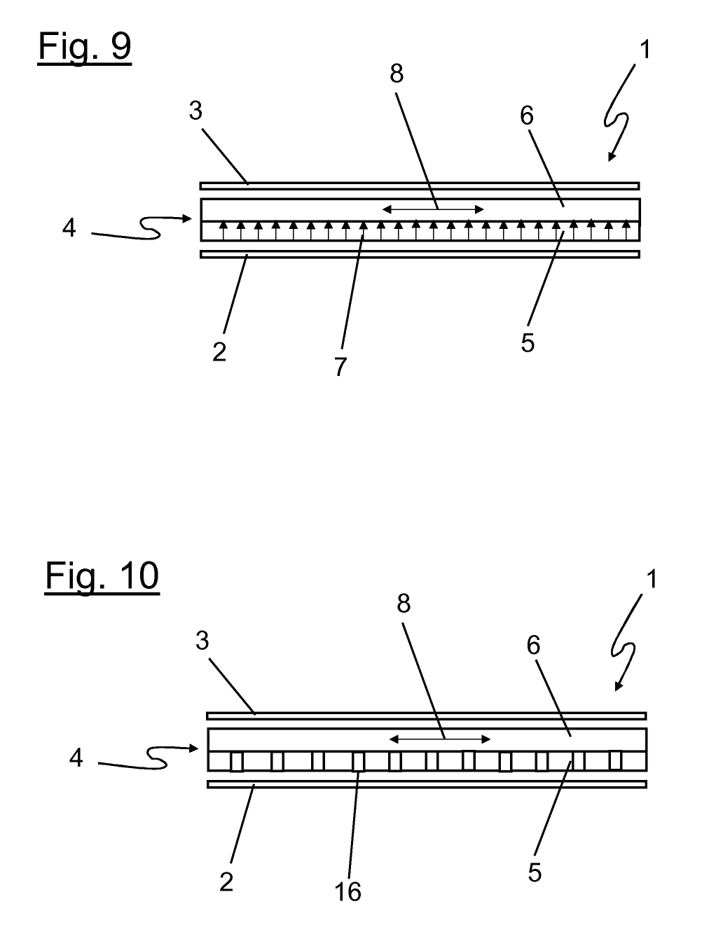

FIG. 9 shows a wound dressing designed for the blood absorption in heavily secreting wounds. The backing layer 3 consists of a hydrophobic non-woven fabric.

As before the core 4 is a two-layered structure comprising a second layer 6 formed of superabsorbent fibers integrated into a non-woven fabric of a mixture of cellulose, viscose and synthetic fibers to absorb blood and to create a horizontal fluid transport.

The first layer 5 of the core 4 not only provides the required vertical transport of fluid, but also serves as a blood absorption layer. Therefore the first layer 5 is made of a non-woven fabric comprising more than 70% of natural cellulose and viscose fibers in order to provide the required blood absorption. As before the fibers in the non-woven fabric of the first layer 5 have been oriented essentially vertically during the needling process. The non-woven fabric used has a density of 0.2 g/cm³ and a weight per area of 150 g/m².

The facing layer has a design which has been described previously with reference to FIGS. 7 and 8.

FIG. 10 shows a schematic drawing of a wound dressing according to a further embodiment of the invention. The design is almost identical to the embodiment shown in FIG. 6. However, the first layer 5 of the core 4 is perforated by a plurality of holes 16 spread all over its surface. In the particular embodiment depicted in FIG. 10 the holes 16 in average have a surface area of 4 mm² The perforation supports the strongly vertically oriented fluid transport properties of the first layer 5.

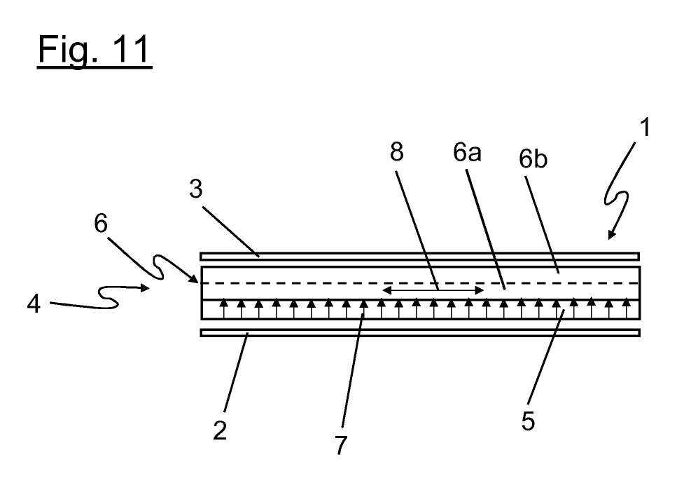

FIG. 11 shows a schematic drawing of an embodiment of a wound dressing according to the invention, wherein the first layer 5 serves to provide a moisture balancing of the wound and a distribution of exudates from the wound. In order to provide these functionalities the first layer 5 is made of purely synthetic material. In the present example the first layer 5 is made of a dry polyester textile. The dry polyester textile comprises hydrophilic PET and bicomponent fibers bonded to form a web. The weight of the first layer 5 is 80 g/m² (average value) and its density amounts to 0.3 g/cm³. The values chosen for the weight and density of the first layer 5 are such that the required functionalities of the first layer 5 are well supported. The first layer 5 is free of any superabsorbent substances.

In a variation of the embodiment depicted in FIG. 11 (not depicted) the first layer could additionally provide absorption to some extend. In order to do so it should be made of a mixture of a synthetic material and a cellulose based material. As a rule of thump the higher the concentration of the cellulose based material is the better the absorption of the first layer will be. Under some circumstances the first layer of this embodiment could also be made of purely cellulose based material.

In the particular embodiment depicted in FIG. 11 the second layer 6 is divided into a set of sublayers 6a, 6b as described before with respect to the embodiment of FIG. 8. The first sub-layer which is nearest to the first layer 5 consists of 100% of cellulosed based fibers. In the example shown in FIG. 11 the first sub-layer 6a of the second layer 6 is made of tissue paper having a weight of 60 g/m². Exudates which have passed through the first layer 5 of the embodiment depicted will be distributed by this arrangement of the first sub-layer of the second layer 6 predominantly in a direction parallel to the facing layer. In the first sub-layer 6a absorption occurs to some extend.

The second sub-layer 6b comprises a superabsorbent substance, in the present case superabsorbent particles, having a weight of 400 g/m² embedded in tissue paper forming a composite material having a weight of 470 g/m² (superabsorbent particles and tissue paper).

In an embodiment having a design similar to the one shown in FIG. 11 a further third sub-layer 6c is provided (not shown). The third sub-layer has the furthest distance from the first layer 5. The design of the third sub-layer 6c is identical to that of the second sub-layer 6b. In particular the second and third sub-layers 6b, 6c are designed such that in combination with each other they do provide the same properties as the single second layer 6b of FIG. 11. Accordingly the second and third sub-layers 6b, 6c comprise a superabsorbent substance, in the present case superabsorbent particles, having a weight of 200 g/m² each embedded in tissue paper forming a composite material having a weight of 235 g/m² each (superabsorbent particles and tissue paper). Thus the combination of the second sub-layer 6b and the third sub-layer 6c is such that together they do comprises superabsorbent particles having a weight of 400 g/m² embedded in tissue paper forming a composite material having a weight of 470 g/m².

As described above the purpose of the third sub-layer 6c is to provide an anti-slumping effect. In addition the third sub-layer 6c of the second layer 6 provides an improvement of the absorption properties of the second layer 6 as between the sub-layer 6b, 6c a wicking in a direction parallel to the extension of the facing layer 2 is provided between the faces of the two layers 6b, 6c.

As described before the backing layer 3 of the embodiment according to FIG. 11 consists of a non-woven fabric comprising a hydrophobic treatment, while the facing layer 2 consists of a polypropylene spunbond nonwoven.

The core of the wound dressing formed by the first layer 5 and the second layer 6 of FIG. 11 provides a fluid distribution to avoid maceration and to get a uniform distribution, absorption and retention (lock fluid) into the whole core area 5, 6. The retention or absorption of al exudates with the bio burden included is necessary to extract it from the wound area and lock it inside the core 5, 6.

The retention of exudates is a key feature of superabsorbent substances, in particular of super absorbent particles. While superabsorbent substances have a high absorption of water their absorption of blood is rather poor compared to natural materials as for example cellulose or more generally speaking cellulose based material. In particular the inherently poor absorption properties of the superabsorbent particles with respect to blood and heavy exudates are enhanced by the distribution of the exudates predominantly parallel to the facing layer 2. By providing this transport of the blood and heavy exudates in a direction parallel to the facing layer the same amount of blood/heavy exudates is spread over a larger area of material containing superabsorbent particles. This in turn effectively leads to better absorption properties of the core 5, 6 with respect to blood and heavy exudates.

The softness of the first layer 5 gives a good contact with the wound which enhances a uniform distribution of exudates together with the second layer 6 over the whole surface which in the next stage gives a proper moisture balance such that the wound is not too wet or dry in a specific area depending on the exact design of the dressing and such that the second or even third sub layer in layer 6 lock the bio burden into the core.

A design wherein the first layer provides a moisture balancing the first layer 5 will not dry out a low exudation wound. In many constructions, superabsorbent particles themselves dry out the wound, when the moisture balance layer not is there. According to an aspect of the invention the superabsorbent particles have a distance to the wound and can just retain what is distributed by the moisture balance layer and the sub layer of cellulose based fibers.

The overall shape of the designs described in detail with reference FIGS. 5 to 11 are various. The dressings may be square, rectangular or circularly shaped as required for application to different parts of a human or animal body. Slitted designs will be available in order to provide dressings in connection with treatment of patients having a tracheotomy.

For purposes of original disclosure it is pointed out that all features which are apparent for a person skilled in the art from the present description, the figures and the claims, even if they have only be described with further features, could be combined on their own or together with other combinations of the features disclosed herein, if not excluded explicitly or technically impossible. A comprehensive explicit description of all possible combinations of features is only omitted in order to provide readability of the description.

1. A wound dressing comprising:
a backing layer;
a facing layer which is permeable for wound exudate and blood and which, when in use, is in contact with the wound; and
an absorbent core;
wherein the backing layer and the facing layer are joined together in order to form a pouch; and
wherein the absorbent core is located within the pouch, wherein the absorbent core comprises at least a first layer and a second layer;
wherein the first layer is located closer to the facing layer than the second layer;
wherein the first layer is configured to provide a flow of liquid predominantly in a direction perpendicular to the extension of the facing layer, and wherein the second layer is configured to provide a flow of liquid predominantly in a direction parallel to the extension of the facing layer.

2. The wound dressing of claim 1, wherein the second layer comprises a first sub-layer and a second sub-layer;
wherein the first sub-layer of the second layer is closer to the first layer than the second sub-layer of the second layer;
wherein the first sub-layer comprises a cellulose based material; and
wherein the second sub-layer comprises a mixture of a cellulose based material, wherein the mixture of a cellulose based material comprises a cellulose based fibrous material and a superabsorbent material, or a mixture of synthetic fibers and a superabsorbent material.

3. The wound dressing of claim 2, wherein the first sub-layer in its unwetted state has a weight in a range from 15 g/m² to 400 g/m²;
wherein the superabsorbent material of the second sub-layer in its unwetted state has a weight in a range from 40 g/m² to 500 g/m²; and
wherein second sub-layer including the superabsorbent material in its unwetted state has a weight in a range 100 g/m² to 800 g/m².

4. The wound dressing of claim 2, wherein the second layer comprises at least a third sub-layer, wherein the third sub-layer has the same properties as the second sub-layer.

5. The wound dressing of claim 1, wherein the first layer consists of synthetic material only, and wherein the first layer is free of any superabsorbent substance.

6. The wound dressing of claim 1, wherein the first layer in its unwetted state has a weight in a range from 12 g/m² to 150 g/m².

7. The wound dressing of claim 1, wherein the first layer in its unwetted state has a density in a range from 0.03 g/m² to 0.4 g/m².

8. The wound dressing of claim 1, wherein the wound dressing further comprises a moisture balance layer located between the absorbent core and the facing layer, wherein the moisture balance layer comprises a non-woven material consisting of synthetic or natural fibers, wherein the fibers of the non-woven material are oriented so that they predominantly extend in a direction perpendicular to the extension of the moisture balance layer.

9. The wound dressing of claim 8, wherein the moisture balance layer is a blood absorption layer containing more than 70% by weight of natural fibers.

10. The wound dressing of claim 8, wherein the moisture balance layer in its unwetted state comprises a density in a range from 0.05 g/cm² to 0.4 g/cm².

11. The wound dressing of claim 8, wherein the moisture balance layer in its unwetted state comprises a weight in a range from 12 g/m² to 150 g/m².

12. The wound dressing of claim 8, wherein the moisture balance layer in its unwetted state comprises a weight in a range from 30 g/m² to 80 g/m² and the moisture balance layer further comprises a superabsorbent substance which in its unwetted state has a weight in a range from 10 g/m² to 30 g/m².

13. The wound dressing of claim 1, wherein the facing layer is made of a material from at least one material selected from a group consisting of a non-woven fabric, a perforated film, alginate, and a foam based on polyurethane or silicone.

14. The wound dressing of claim 1, wherein facing layer comprises a non-woven fabric consisting of synthetic or cellulose fibers, wherein the fibers of the non-woven fabric are oriented such that they predominantly extend in a direction perpendicular to the extension of the facing layer.

15. The wound dressing of claim 1, wherein the first layer is perforated by a plurality of holes, wherein the holes have a diameter in a range from 1.5 mm to 5 mm.

16. The wound dressing of claim 1, wherein the facing layer is perforated by a plurality of holes, wherein the holes have a diameter in a range from 1.5 mm to 5 mm.

17. The wound dressing of claim 8, wherein the moisture balance layer is perforated by a plurality of holes, wherein the holes have a diameter in a range from 1.5 mm to 5 mm.
